(19) United States
(12) Patent Application Publication
(10) Pub. No.: US 2016/0213714 A1
(43) Pub. Date: Jul. 28, 2016

(54) INDUCTION OF CELLULAR SENESCENCE FOR TISSUE THERAPIES

(71) Applicants: Everon Biosciences, Inc., Buffalo, NY (US); Health Research, Inc., Buffalo, NY (US)

(72) Inventors: Andrei V. Gudkov, East Aurora, NY (US); Olga Chernova, Orchard Park, NY (US); Brandon Hall, Buffalo, NY (US)

(21) Appl. No.: 15/005,518

(22) Filed: Jan. 25, 2016

Related U.S. Application Data
(60) Provisional application No. 62/107,152, filed on Jan. 23, 2015.

(51) Int. Cl. A61K 35/13; A61L 27/38; A61L 27/36; A61L 27/54

(52) U.S. Cl. CPC A61K 35/13; A61L 27/54; A61L 27/3804; A61L 27/3687; A61L 2300/64; A61L 2300/412; A61L 2300/30

(57) ABSTRACT

Provided are compositions and methods for aiding in healing of a tissue wound in an individual. The method generally entail delivering to an individual in need thereof senescent cells such that healing of the wound is accelerated. The disclosure includes use of senescent cells and senescent tumor cells, either of which may be allogeneic or autologous to the individual. Also provided are methods for inducing senescence in cells and using the cells for wound healing, and articles of manufacture comprising senescent cells for use in wound healing.

### CROSS REFERENCE TO RELATED APPLICATIONS

This application claims priority to U.S. provisional application No. 62/107,152, filed Jan. 23, 2015, the disclosure of which is incorporated herein by reference.

### FIELD

The present disclosure relates generally to regenerating tissue, and more specifically to the use of senescent tumor cells as a source of new stroma for use in tissue repair.

### BACKGROUND

Healing of various types of acute and chronic wounds with full and rapid restoration of tissue integrity and functionality is an important medical need and a significant market. Some current approaches use normal mesenchymal cells (MSCs) for stimulation of tissue regeneration and wound healing. Such approaches for the regeneration of damaged tissues typically involve administration of cells of mesenchymal origin (usually in combination with matrix scaffolds and growth hormones) which directly participate in and/or promote the deposition of extracellular matrix and angiogenesis (WO 2008004260 A2; WO2002017980 B1; U.S. Pat. No. 6,355,239 B1). The potential therapeutic use of primary cultures of mesenchymal stem cells (MSCs) for stromagenesis has been described for soft tissue damage and/or vascular insufficiency which result from acute trauma (surgery, irradiation, burns, etc.) and/or disease (diabetes, ischemia, chronic wounds, etc.) (EP0953040 A1; WO2007149861 A2; US20100233134 A1; EP2037963 A2; US20120100114 A1). Other approaches using MSCs involve either stimulation with bioactive molecules or genetic engineering in order to enhance efficacy. Examples include activation of MSCs via inflammatory cytokines to promote stromal development (U.S. Pat. No. 8,119,398 B2; US20130017175 A1) or overexpression of receptors which enhance angiogenic potential (U.S. Pat. No. 8,617,883 B2). However, many of the foregoing approaches have met little or only limited success. There is therefore an ongoing and unmet need for new approaches to tissue repair in a wide variety of settings.

### SUMMARY OF THE DISCLOSURE

The present disclosure provides in various embodiments a method for aiding in healing of a tissue wound in an individual. The method generally comprises delivering to the individual senescent cells such that healing of the wound is accelerated. In certain approaches the cells are delivered directly to the wound. In one non-limiting example, the senescent cells are senescent tumor cells, and may be allogeneic to the individual, or autologous to the individual. In one approach, the disclosure comprises, prior to delivering the senescent cells, exposing non-senescent cells to a senescence inducing agent to thereby obtain the senescent cells. In certain implementations delivering senescent tumor cells promotes formation of a pseudo-tumor. The pseudo-tumor may comprise cells of the individual that are recruited to the pseudo-tumor by the senescent tumor cells.

The type of tissue wound that can be treated is not particularly limited, and includes incisions and other forms of cuts, burns, ulcerations, and ischemic injury. In cases of ischemic injury, it can occur in any tissue and for any reason, including but not limited to myocardial infarction, renal ischemia, and liver ischemia. Likewise, the type of individual treated according to embodiments of this disclosure is not particularly limited, and includes human and non-human animals, such as non-human mammals.

In certain approaches compositions and methods of this disclosure are used during or soon after a surgical procedure, including but not limited to a tissue or organ transplantation. In certain approaches the individual is a non-human animal and is undergoing or is recovering from a xenograft procedure.

In certain examples the senescent cells or the senescent tumor cells secrete pro-inflammatory factors and stimulate an immune response and/or attract macrophages to the wound to facilitate wound healing.

In one aspect the disclosure includes providing senescent cells and/or senescent tumor cells in a composition comprising a biocompatible surface, wherein at least some of the senescent cells and/or the senescent tumor cells are in physical association with the biocompatible surface. Such embodiments include combinations with a heterologous or autologous engraftment material. Combinations of the senescent cells and/or the senescent tumor cells and the biocompatible surface and/or the engraftment material can be delivered to the individual concurrently. In certain examples the biocompatible surface comprises a natural or synthetic matrix or a plurality of particles.

The disclosure includes an article of manufacture at least one container, the container comprising a composition comprising senescent cells or senescent tumor cells, the container or packaging material comprising the container comprising printed information, the printed information providing an indication that the senescent cells or the senescent tumor cells are for use in wound healing in an individual.

The disclosure also includes methods for making senescent cells for use in wound healing. In certain implementations the method comprises exposing non-senescent cells to a senescence-inducing agent, and subsequent to inducing senescence in the cells, administering the senescent cells to an individual in need of wound healing. In certain approaches the senescent cells can be combined with an engrafting material prior to the administering.

### BRIEF DESCRIPTION OF FIGURES

FIG. 1: Senescence-associated beta-galactosidase (SA-β-Gal) activity of bleomycin-treated cells. A549 cancer cells (panel A) and primary human WI-38 fibroblasts (panel B) were treated for 72 hours with 50 ug/ml or 15 ug/ml of bleomycin, respectively. Cells were fixed and stained 7 days post-bleomycin treatment. Non-treated cultures, confluent at the time of staining, were used as controls. Enlarged cellular morphology of senescent cells was also observed (images to scale).

FIG. 2: Average tumor size of A549-GFP senescent and proliferating xenografts in SCID mice. Indicated number of bleomycin-induced senescent and/or non-treated cells (in millions, M) were injected into the flanks of mice and grown for 20 days. Groups consist of 2 mice (4 inoculations total), except for ‘10M SenBleo’ where only 1 mouse was used (2 inoculations total). The mouse injected with 10 million senescent cells (10M SenBleo) was sacrificed on day 6 for analysis. Error bars depict standard deviation. Asterisks indicate statistical significance as determined by two-tailed student's t-test (p<0.05; 1M Non-treated versus SenBleo groups).

FIG. 3: Senescence-associated beta-galactosidase (SA-β-Gal) activity of A549-GFP xenograft pseudo-tumors. Bleomycin-induced senescent A549-GFP cancer cells (107 cells) were inoculated into the flanks of SCID mice. Pseudo-tumors were collected on Day 6 at an average tumor volume of 153 mm3. Pseudo-tumors were sectioned and stained for SA-beta-Gal activity with nuclear fast red counterstain (upper left: 2.5× objective; lower left: 20× objective), stained for GFP expression (upper right), and stained with H&E counterstain (lower right). A large necrotic region can be observed in the center of the pseudo-tumor mass (lower right in each image at 2.5× objective), likely due to the rapid growth of the pseudo-tumor as the volume doubled in size from day 4 to day 5 (from 88 to 166 mm3).

FIG. 4: Immunohistochemical analysis of senescent xenograft pseudo-tumors. Pseudo-tumors derived from injecting 10 million senescent A549-GFP cells were stained for Collagen I (red), with DAPI (blue) counterstain to label nuclei. Higher magnification images of pseudo-tumor regions of necrosis (bottom left) or living tissue from the pseudo-tumor periphery (bottom right) are depicted.

FIG. 5: Immunohistochemical analysis of senescent xenograft pseudo-tumors. Pseudo-tumors derived from injecting 10 million senescent A549-GFP cells were stained for Collagen IV (red) and PECAM (green) on the left panel, or Collagen III (red) and CD146 (green) on the right panel. Both panels show DAPI (blue) counterstain to label nuclei. Images are from pseudo-tumor periphery.

FIG. 6: Immunohistochemical analysis of senescent xenograft pseudo-tumors. Pseudo-tumors derived from injecting 10 million senescent A549-GFP cells were stained for smooth muscle actin (SMA) (red) and CD105 (green), with DAPI (blue) counterstain to label nuclei. The imaged pseudo-tumor periphery (upper left) is also depicted in higher magnification (lower and right panels).

FIG. 7: Immunohistochemical analysis of senescent xenograft pseudo-tumors. Pseudo-tumors derived from injecting 10 million senescent A549-GFP cells were stained for Vimentin (red; lower left panel) and Nestin (green; lower right panel), and with DAPI (blue; upper right panel) counterstain to label nuclei. An image of the merged channels of pseudo-tumor periphery is depicted (upper left panel).

FIG. 8: Average pseudo-tumor size of A549-GFP senescent and proliferating xenografts in SCID mice. Indicated senescent (bleomycin-induced senescence, SenBleo; busulfan-induced senescence, SenBU) or proliferating (Prolif) cells were injected into the flanks of SCID mice (4 million cells per injection). Groups consist of 2 pseudo-tumors. Error bars depict standard deviation. Statistical differences (via two-tailed t-test; p <0.05) was observed between SenBU versus Prolif groups on days 2 and 4, and in SenBU and SenBleo versus Prolif groups on days 16 and 18.

FIG. 9: Pseudo-tumor size of primary WI-38 senescent and proliferating xenografts in SCID mice. Indicated senescent (bleomycin-induced senescence, SenBleo) or proliferating (Prolif) cells were injected into the flanks of SCID mice (4 million cells per injection). Groups consist of 1 inoculation site.

FIG. 10: Pseudo-tumor size of WI-38 senescent and proliferating xenografts in SCID mice. Senescent (bleomycin-induced senescence, SenBleo) or proliferating (Prolif) cells as indicated were injected into the flanks of SCID mice (1.2 million cells per injection). Cells were injected with or without pre-treatment of 0.1% sodium azide (+NaN₃). Groups consist of 1 inoculation site.

FIG. 11: Immunostimulatory effects of secretory factors produced by senescent cells: comparison of immunocyte populations attracted to i.p.-injected alginate beads carrying senescent and non-senescent human neonatal dermal fibroblasts (NDF). (A) Senescent (following 20 Gy gamma-irradiation) and non-senescent (mock irradiation, 0Gy) control human neonatal dermal fibroblasts (NDFs) were embedded in polymerized alginate (biologically inert polysaccharide) beads (˜0.5 mm diameter; containing 5 million cells/mL), which protected embedded cells from a direct contact with immune cells, but allows release of secreted factors (i.e. SASP factors) and cell access to oxygen and nutrients. Staining for SA-beta-Gal activity of beads prior to injection demonstrated strong staining of senescent cells as compared to beads containing control fibroblasts (left column panels). Alginate beads were injected (0.3 mL packed bead volume) into the peritoneal cavity of NIH-Swiss mice. After 18 days, beads were retrieved from mice and analyzed under microscope. Significantly higher number of cells were found adherent to the surface of senescent cell-containing beads than to the beads carrying non-senescent cells. Viability of beads-adherent cells was determined using calcein-acetoxymethyl (calcein-AM) dye producing green fluorescent staining in live cells (right column).) (B) Quantification of macrophages present in 5 mL of peritoneal lavage and of cells attached to 100 ul of packed alginate bead volume. The host mouse cells were removed from the bead's surface by collagenase treatment and characterized by flow cytometry using a set of antibodies against cell type-specific biomarkers. The number of peritoneal macrophages in lavage fluid and in population of beads-attached cells was determined in viable CD45+ cells using F4/80 marker. Almost six times more macrophages were found attached to senescent cell-containing alginate beads then to control NDF-containing alginate beads.

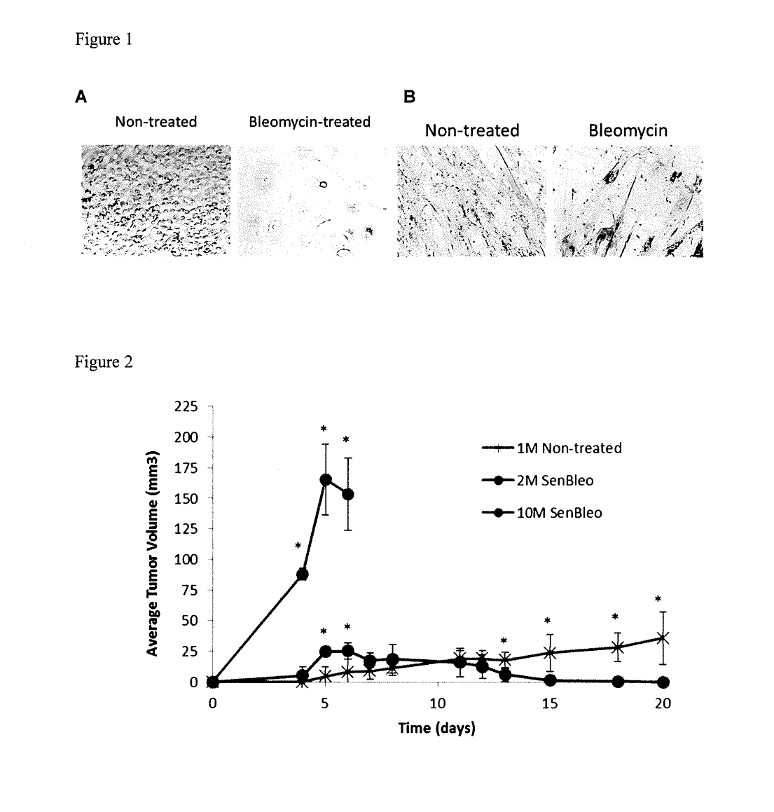

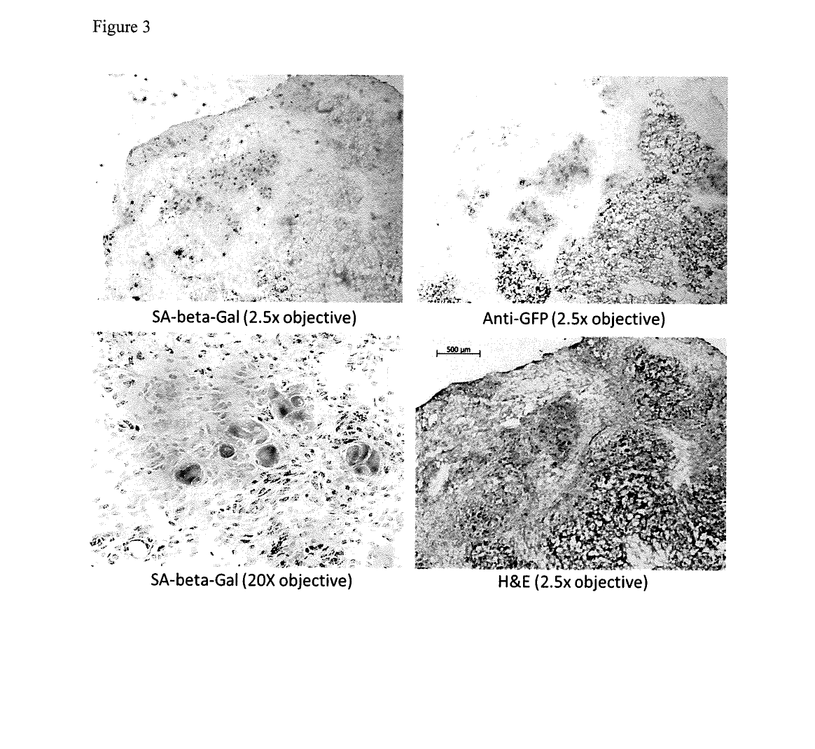

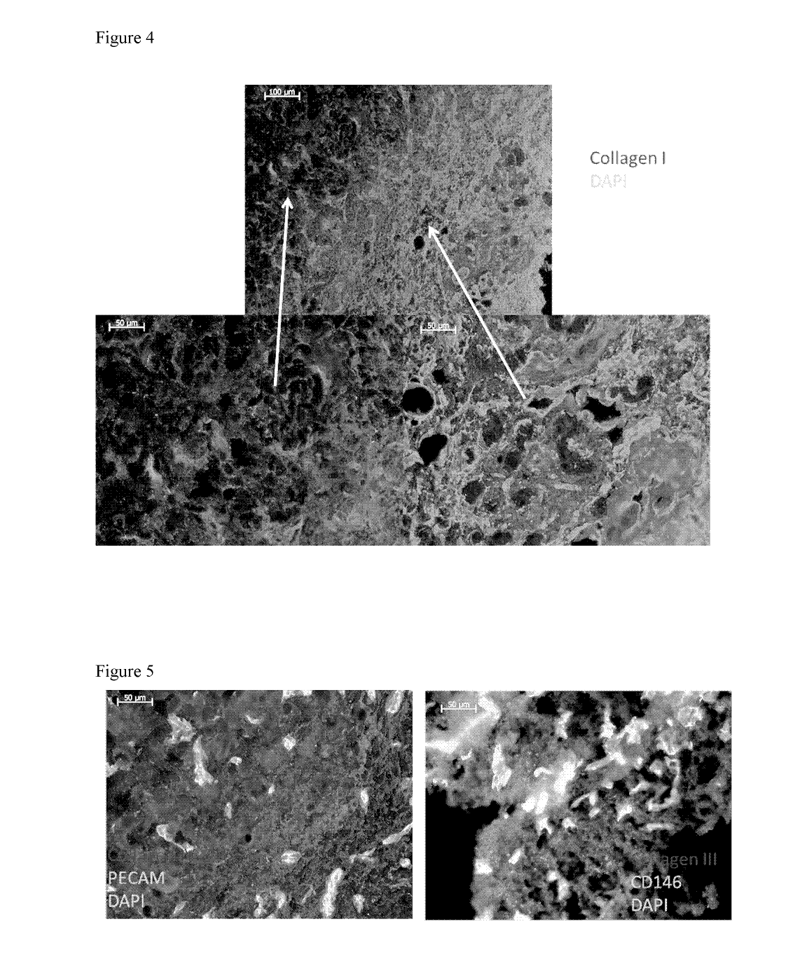

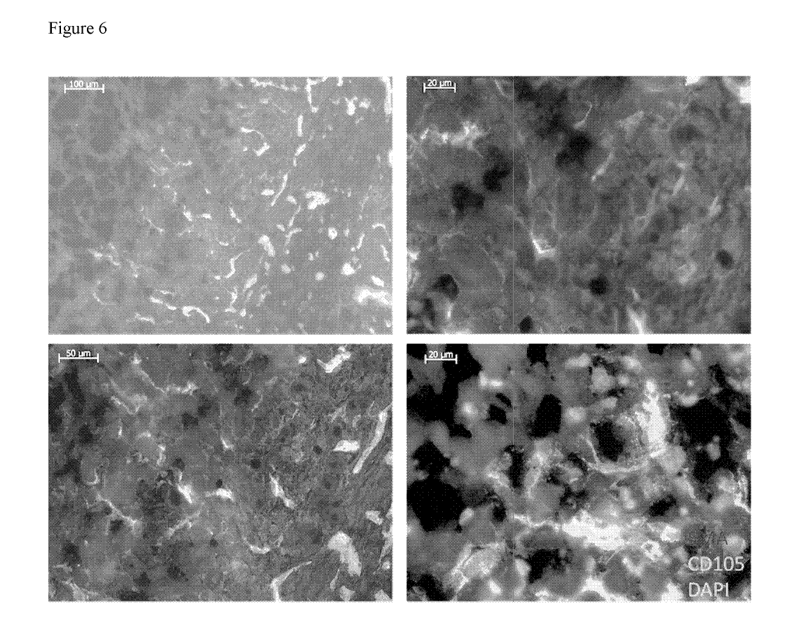

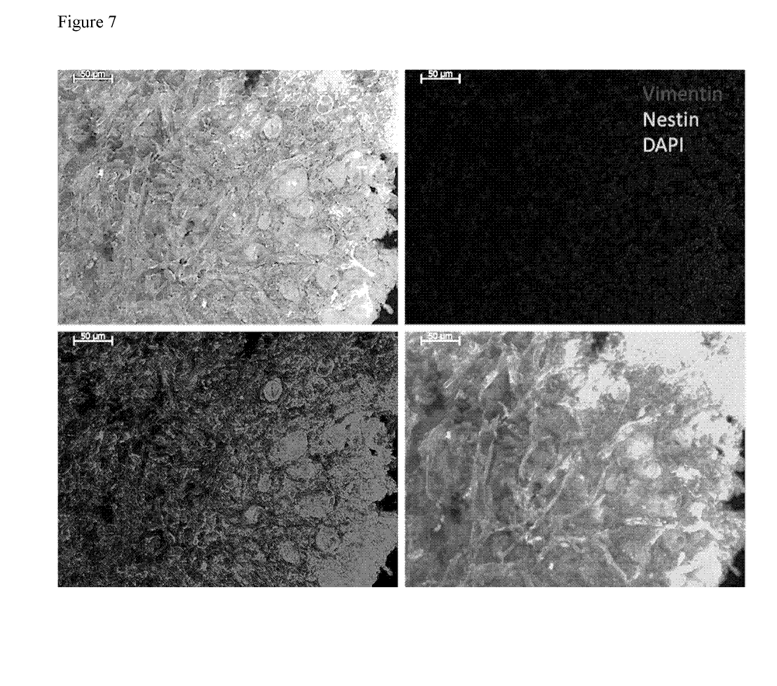

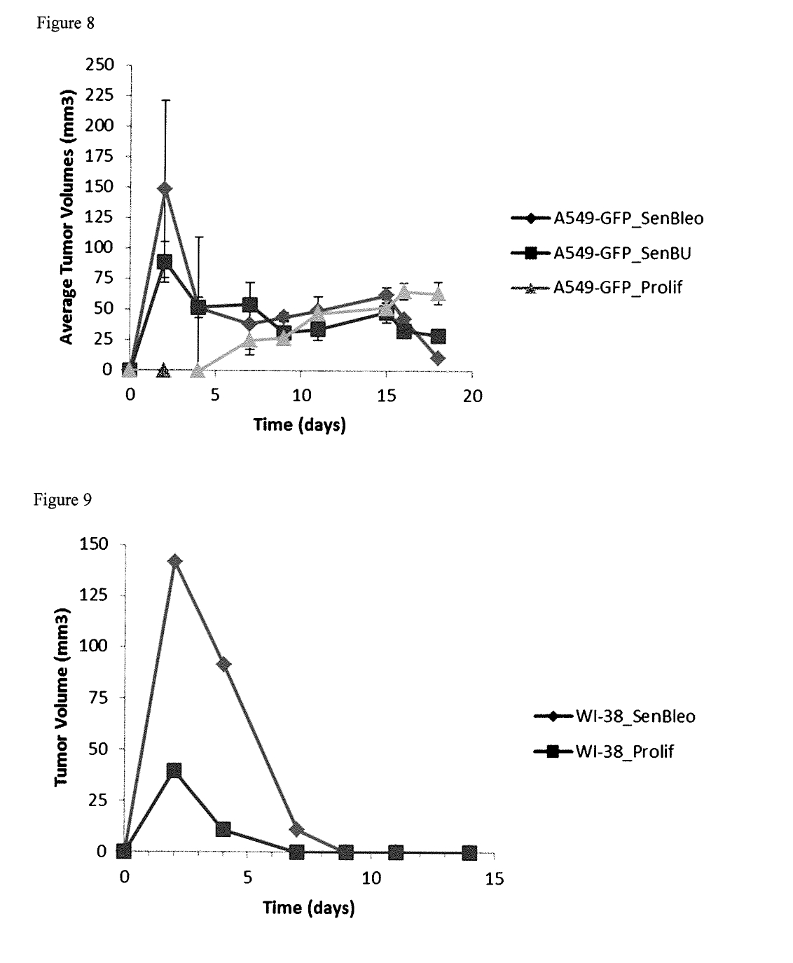

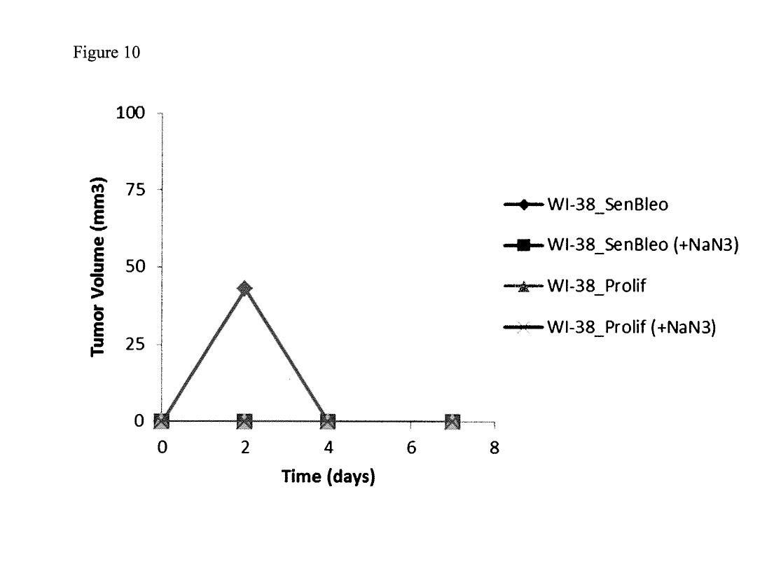

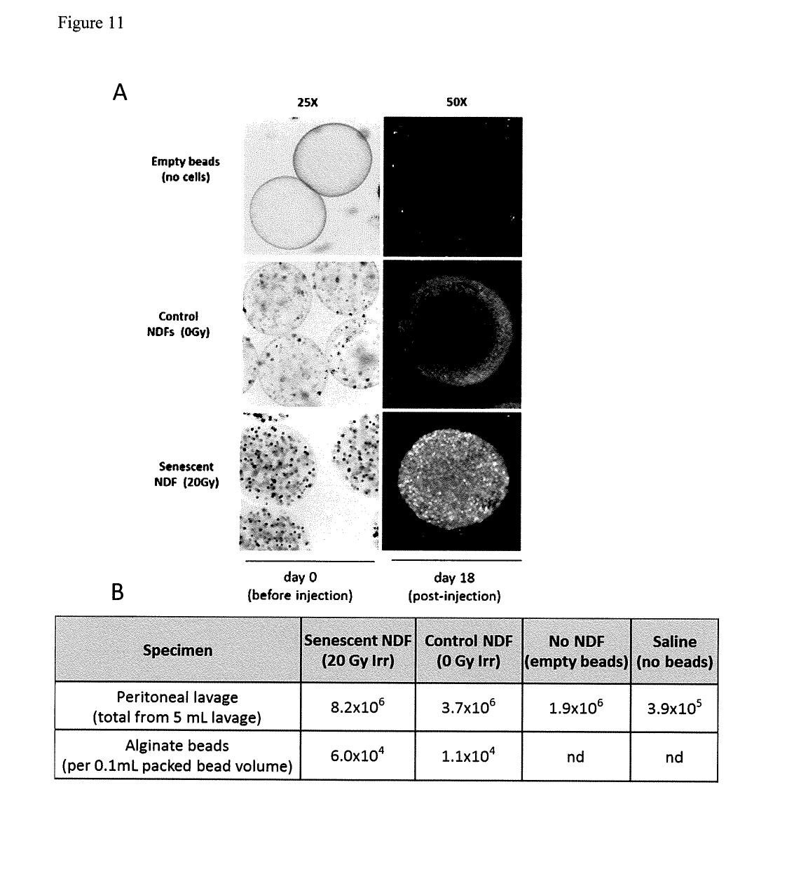

### DETAILED DESCRIPTION

The present disclosure is related generally to the discovery that previous approaches to tissue repair/regeneration, which use normal (i.e., non-malignant/transformed) mesenchymal cells may have had only limited success because such normal mesenchymal cells do not exhibit robust angiogenic or stromagenic capabilities. Further, such normal cells predominantly make up the material for tissue infrastructure restoration, rather than attracting and stimulating cells for self-renewal. In contrast, as described more fully by way of the following description and figures, the present disclosure reveals that tumor cells which have undergone an epigenetic transition to a state of cellular senescence exhibit a strong capability to attract and mobilize various factors and cell types that are important for tissue regeneration. Thus, in general, the present disclosure provides methods for aiding in tissue repair comprising delivery to an individual in need of senescent cells, such that the senescent cells facilitate tissue repair in the individual, by for example, angiogenesis and/or stromagenesis. In embodiments, the senescent cells are senescent tumor cells.

In particular, the present disclosure reveals that during the progression to a malignant phenotype, tumor cells undergo natural selection for angiogenic and stromagenic properties in order to attract endogenous cells to the sites that require regeneration. However, these aspects of tumor cells are greatly enhanced after the tumor cells undergo a transition to senescence. These are unexpected and useful properties that have not been previously described. Thus, the present disclosure is based at least in part on the surprising discovery that senescent cells, including senescent tumor cells, can be exploited for beneficial purposes in a wide variety of settings where repair and/or regeneration of tissue is desired. These and other advantageous features of the compositions and methods of this disclosure include but are not necessarily limited to: i) the conversion to an irreversible senescent phenotype renders tumor cells safe, since they cannot revert to a malignant state; ii) the strength of the stromagenic effect exhibited by senescent tumor cells is enhanced (relative to non-senescent cells) that it eliminates the requirement for the cells to be syngeneic with the patient, delaying any deleterious immunological response until after they have served their purpose (i.e., promoting angiogenesis and attracting cells to form new stroma at and/or near a location in in need of the angiogenesis and/or the new stroma). Further, data presented in this disclosure demonstrate the stromagenic effect is brought about rapidly, i.e., within three days post introduction of the senescent tumor cells into a recipient. Thus, these and other features that will be apparent to those skilled in the art from the present disclosure makes it plausible to generate a commonly usable product comprising irreversibly senescent tumor cells, or other suitable cells, such as senescent mesenchymal cells, or fibroblasts. In an embodiment the senescent tumor cells are used.

Those skilled in the art will recognize a senescent cell refers to the essentially irreversible growth arrest that occurs when cells that can divide encounter oncogenic stress or DNA damage. See, for example, Rodier F, Campisi J. Four faces of cellular senescence. J Cell Biol. 2011 February, from which the description of senescence is incorporated herein by reference. In embodiments, “senescent tumor cells” as the term is used in this disclosure refers to tumor cells which express a marker or combination of markers that are characteristic of senescence. Such markers include but are not necessarily limited to the p16INK4a tumor-suppressor protein, and increased expression relative to a reference, such as a non-senescent cell, in the levels of DNA-damage response (DDR) markers, as well as the cell cycle inhibitors p16^{INK4A}, p15^{INK4B}, p21^{CIP1}, and p53. DEC1, DCR2 (ColladoM, et al. 2005. Tumour biology: Senescence in premalignant tumours. Nature 436: 642), and PALL (GoldsteinS, et al, 1994). Overexpression of plasminogen activator inhibitor type-1 in senescent fibroblasts from normal subjects and those with Werner syndrome. J Cell Physiol 161: 571-579) have also been used as senescence biomarkers (Collado M, Serrano M. 2010. Senescence in tumours: Evidence from mice and humans. Nat Rev Cancer 10: 51-57). In one embodiment, senescent cells are SA-beta-Gal (senescence-associated beta galactosidase) positive. In an embodiment, a senescent phenotype comprises an enlarged cellular morphology.

In certain embodiments, the present disclosure comprises delivering to an individual in need thereof an effective amount of senescent tumor cells such that the senescent tumor cells promote healing of a wound in the individual. In embodiments, the wound comprises an incision or other separation of tissue, or comprises a burn, or comprises a laceration, or an ulceration, including but not necessarily limited to a diabetic ulcer. In embodiments, the wound comprises tissue damage induced by contact with heated objects and/or surfaces, or light, or chemicals, or a wound caused by medical techniques such as surgical interventions wherein the skin, other tissue or an organ is cut or pierced or avulsed, or other non-medical wounds which cause trauma by any means, including but not necessarily to the accidental or intentional wounding of an individual, such as in a military conflict or other act of violence, an industrial accident, a vehicular accident, or an injury sustained during a sporting event. In embodiment, the wound is from hypoxia or ischemia, such as a localized ischemic event. In certain embodiments the disclosure encompasses healing of wounds that are incidental to or a component of organ and/or tissue transplantation. Thus, the present disclosure includes aiding in organ and tissue transplantation by including senescent cells in the transplantation procedure. The senescent cells can be included in the transplant procedure to, for example, enhance healing of the transplanted tissue or organ. In embodiments, the material, tissue, or organ can be mixed with senescent cells. Thus in embodiments, the present disclosure includes compositions suitable for engraftment which can comprise autologous or heterologous engraftment tissue and/or organs, and which further comprise the senescent cells that are described herein.

In embodiments, the present disclosure pertains to use of senescent cells for improving engraftment of xenografts, such as for tumors and normal tissues, that in certain embodiments can be used in non-human animal settings. In embodiments, the present disclosure provides for an in the efficacy of tissue engraftment by mixing engrafted material with senescent cells and engrafting the mixed composition. The present disclosure thus also includes a combination of material intended for engraftment and senescent cells. In embodiments, the material comprises the senescent cells and a biocompatible matrix. In embodiments, by including senescent cells with engraftment material, an increase in the efficacy of foreign tissue engraftment is achieved. In embodiments, the amount of engrafted material that remains viable after the engraftment is increased, and/or the engraftment wound heals faster relative to a wound treated without the senescent cells as part of the engraftment material.

The wound can be to any part of an individual. In embodiments, the wound is in a soft tissue, such as skin, or is in an organ, for example, kidney or heart (myocardium infarction), or a muscle, or bone

The senescent tumor cells or compositions comprising them can be delivered to an individual using any suitable technique. In embodiments, the cells are administered subcutaneously, intravenously, intra-arterially, or are injected directly into other wounded tissue. In other embodiments, the cells are provide as a component of or are introduced into a scaffolding material. In embodiments, the scaffolding material comprises a porous surface, such as a sponge-like material or other synthetic or natural biocompatible matrix, such as a collagen matrix. In embodiments, the cells are provide as a component of a composition that comprises a carrier, such as a biocompatible nano-particle formulation, polymeric beads, a biocompatible mesh, and the like. In embodiments, the cells are administered by implantation.

Given the benefit of the present disclosure, those skilled in the art will be able to determine an effective amount of senescent cells to deliver. Such determinations will be based on factors that can include but are not limited to the size, age and type of individual to be treated, and the type, size, severity, and location of the wound or other circumstance that is to be treated according to this disclosure. In embodiments, from 10,000 up to 20,000,000, including all integers and ranges there between, senescent cells are introduced to the individual.

In certain embodiments, the method comprises delivering senescent tumor cells to an individual to aid in the healing of an organ or other tissue transplantation.

In embodiments, the method of the invention results in accelerated healing of soft tissue wounds relative to healing in a control, i.e., relative to a case wherein senescent cells are not given to the individual.

In embodiments, the method of the disclosure comprises introducing senescent tumor cells into an individual such that a pseudo-tumor is formed. A pseudo-tumor is a viable mass formed in the individual, wherein the majority of the mass is not comprised of the senescent tumor cells. Thus, the viable mass can comprise mostly stroma and/or non-senescent-tumor cells, such as cells of mesenchymal and/or endothelial origin. In embodiments, the pseudo-tumor is transient. In embodiments, the pseudo-tumor persists for less than four weeks, three weeks, or two weeks in the individual.

In distinct embodiments, the senescent cells introduced into an individual are obtained or derived from the individual, or are obtained or derived from a different individual. Thus, the disclosure includes allogeneic and syngeneic approaches. In embodiments, senescent tumor cells are used, and are propagated as a cell line for use in methods of this disclosure. Accordingly, such in vitro cultured cells and their progeny are encompassed in this disclosure.

In embodiments, the compositions and methods described herein are suitable for use with any mammal in need thereof. The mammal can be a human or a non-human mammal. Thus, in addition to human medicaments and treatment modalities, the present disclosure also encompasses veterinary aspects for the treatment of, for example, companion animals, livestock, etc.

In embodiments, the disclosure includes making senescent cells for use in stromagenesis, angiogenesis, and other methods described herein using any suitable starting cells and techniques and reagents for converting non-senescent cells to a senescent phenotype. The choice of tumor cells depends on the particular wound healing/tissue regeneration need. It is expected that any tumor cell type can be suitable for this purpose, provided it is prone to undergoing senescence following a DNA damaging treatment, thus rendering the cells incapable of proliferation. For such conversion of the tumor or other cancer cells to a senescent state, one can use, for example, sterilizing doses of ionizing radiation. Without intending to be constrained by any particular theory, it is expected that the capability to senesce in tumor cells is associated with retention of wild type and partially functional p53. Rapidly growing p53-wild type tumor cells are considered to be particularly effective in their tissue regeneration induction capabilities. It is expected that tissue origin of the tumor will predict the wound healing preference of its senescent derivatives. Tumor cells suitable for such use include but are not necessarily limited to: lung, breast, basal cell, cervical carcinoma, melanoma, etc.

In general, to coax development of senescence, non-senescent cells are exposed to a reagent that is known to induce DNA damage or to have other genotoxic properties, such that subsequent to the exposure to the reagent, the cells become senescent. In embodiments, the cells are exposed to a compound that induces a break in a single or both DNA strands, or causes cross-linking of DNA strands, or otherwise interferes with DNA replication but is not lethal to the cells. In an embodiment, the DNA strand breaking agent is bleomycin, i.e., bleomycin A2 or B2. In an embodiment, the DNA cross-linking agent is an intrastrand cross-linking agent. In an embodiment, the intrastrand cross linking agent is an alkyl sulfonate, such as busulfan. Other methods of inducing senescence in tumor cells will be apparent to those skilled in the art and include but are not limited to the use of chemotherapeutic agents, such as antibiotics, such as the aforementioned bleomycin, or doxorubicin, or intercalating agents, or mitomycins such as mytoycin C, or a topoisomerase agent such as etoposide. Biophysical treatments leading to DNA-damage-mediated senescence include the use of ionizing radiation or ultraviolet light.

In certain aspects the disclosure comprises selecting an individual having a wound in need of treatment to receive senescent cells as an approach to healing the wound. In certain aspects the disclosure includes delivering the senescent cells and monitoring wound healing in an individual who received the senescent cells. Wound healing may occur in conjunction with monitoring one or more indicia associated with the senescent cells that were delivered to the individual, including but not necessarily limited to determining the location, morphology, and/or biochemical properties of the senescent cells, such as secretion of compounds that stimulate immune response, and/or attract other cells to the wound site, including but not limited to macrophages. In certain aspects the cellular composition of the wound site comprises an increased amount of immune cells relative to a control. In certain approaches the senescent cells secrete pro-inflammatory factors which stimulate an immune response, which may comprise attracting macrophages to the wound site and to facilitate wound healing. In certain embodiments the pro-inflammatory factors comprise one or more pro-inflammatory cytokines, including interleukins, including but not necessarily limited to IL-1, IL-6 and IL-8. Also included are TNF alpha and interferon gamma.

In an embodiment, the disclosure includes a composition comprising senescent tumor cells. In embodiments, the compositions comprises a pharmaceutically acceptable buffer, excipient or carrier, and thus can be considered a pharmaceutical or biological agent composition.

In one embodiment, the disclosure includes an article of manufacture. In certain aspects, the article of manufacture includes a closed or sealed container, and packaging, that contains senescent tumor cells as described herein. The package can include one or more containers, such as closed or sealed vials, bottles, and any other suitable packaging for the sale, or distribution, or use of pharmaceutical or biologic agents. Thus, the package can contain pharmaceutical or biologic compositions which contain any senescent cells, and optionally other compounds that are described herein. In addition to the pharmaceutical or biologic compositions, the package and/or container may contain printed information. The printed information can be provided on a label, or on a paper insert, or printed on the packaging material or container itself. The printed information can include information that identifies the types of cells, and/or an indication of what condition(s) the contents is intended to treat, and instructions for preparing the composition for administration, and/or for administering the composition, such as the number and type of senescent cells in the container, and what amount to of cells deliver to the individual in need. Thus, in various embodiments the disclosure includes a pharmaceutical/biologic composition of the invention packaged in a packaging material and identified in print, on or in the packaging material, that the composition is for use in the treatment of wounds, or for organ implantation, or for angiongenesis, or for stromagenesis, or for accelerating the healing of soft tissue wounds, or combinations thereof.

Although the embodiments have been described in detail for the purposes of illustration, it is understood that such detail is solely for that purpose, and variations can be made therein by those skilled in the art without departing from the spirit and scope of the disclosure, embodiments of which are defined by the following sample claims.

What is claimed is:

1. A method for aiding in healing of a tissue wound in an individual comprising delivering to the individual senescent cells such that healing of the wound is accelerated.

2. The method of claim 1, wherein the senescent cells are senescent tumor cells.

3. The method of claim 2, wherein the senescent tumor cells are allogeneic to the individual.

4. The method of claim 2, wherein the senescent tumor cells are autologous to the individual.

5. The method of claim 1, comprising prior to delivering the senescent cells, exposing non-senescent cells to a senescence inducing agent to thereby obtain the senescent cells.

6. The method of claim 5, wherein the senescent cells delivered to the individual are senescent tumor cells.

7. The method of claim 2, wherein the senescent tumor cells promote formation of a pseudo-tumor, wherein the pseudo-tumor comprises cells of the individual recruited to the pseudo-tumor by the senescent tumor cells.

8. The method of any one of the preceding claims, wherein the wound is an incision, a cut, a burn, an ulceration, an ischemic injury, and/or is created during organ transplantation.

9. The method of claim 8, wherein the wound is an ischemic injury which comprises a myocardial infarction, a renal ischemia, a liver ischemia, or a combination thereof.

10. The method of any one of claims 1-7, wherein the senescent cells or senescent tumor cells are in a composition comprising a biocompatible surface, and wherein at least some of the senescent cells or the senescent tumor cells are in physical association with a biocompatible surface, and/or wherein the senescent cells or the senescent tumor cells are combined with a heterologous or autologous engraftment material, and wherein the combination of the senescent cells or the senescent tumor cells and the biocompatible surface and/or the engraftment material is delivered to the individual.

11. The method of claim 10, wherein the biocompatible surface comprises a natural or synthetic matrix or a plurality of particles.

12. The method of any one of claims 1-7 wherein the senescent cells or the senescent tumor cells secrete pro-inflammatory factors and stimulate an immune response and/or attract macrophages to the wound to facilitate wound healing.

13. An article of manufacture at least one container, the container comprising senescent cells or senescent tumor cells, the article of manufacture further comprising printed information, wherein the printed information provides an indication that the senescent cells or the senescent tumor cells are for use in wound healing in an individual.

14. A method comprising exposing non-senescent cells to a senescence-inducing agent, and subsequent to inducing senescence in the cells, administering the senescent cells to an individual in need of wound healing.

15. The method of claim 14, wherein the individual in need is an individual undergoing or recovering from a transplant of a tissue or an organ.

16. The method of claim 15, wherein the individual is a non-human animal and is undergoing or is recovering from a xenograft procedure.

17. The method of any one of claims 14-16, wherein the senescent cells are combined with an engrafting material prior to the administering.
